(19) United States
(12) Patent Application Publication
(10) Pub. No.: US 2025/0325769 A1
(43) Pub. Date: Oct. 23, 2025

(54) AN IMPROVED METHOD OF CONVERTING VENOUS BLOOD GAS VALUES TO ARTERIAL BLOOD GAS VALUES

(71) Applicant: Roche Diagnostics Operations, Inc., Indianapolis, IN (US)

(72) Inventor: Bjarne Flou, Kobenhavn V (DK)

(21) Appl. No.: 18/870,898

(22) PCT Filed: Jan. 6, 2023
(86) PCT No.: PCT/EP2023/064693

§ 371 Date: Dec. 2, 2024

(30) Foreign Application Priority Data
Jun. 2, 2022 (EP) 22176990.4

(51) Int. Cl. A61M 16/00; G16H 10/40; G16H 70/20

(52) U.S. Cl. CPC A61M 16/026; G16H 10/40; A61M 2202/0208; G16H 70/20

(57) ABSTRACT

A computer-implemented method, system and decision support system adapted to provide arterial venous blood gas values without the provision of an arterial oxygenation saturation value or arterial blood gas values. The method comprises the provision of arterial blood gas values from a subject, for which said subject, only venous blood gas values are provided, by providing a mathematical model adapted to convert said venous blood gas values with a provided predefined default arterial oxygenation value to output arterial blood gas values of said subject. The present invention thus provides a method for providing arterial blood gas values from a specific subject without the need of providing an arterial blood sample from a painful arterial blood draw or the need for an arterial oxygenation saturation value of the subject, thus reducing distress to said patient and a reduction of tasks to relevant health care personnel.

### FIELD OF THE INVENTION

The present invention relates to an improved method of converting venous blood gas values from a subject to arterial blood gas values of the subject and a corresponding computer program product for executing the method on a computer system. The invention also relates to a corresponding decision support system (DSS), preferably a portable data processing system, and a corresponding computer program product.

### BACKGROUND OF THE INVENTION

The assessment of acutely ill patients is a complex process involving evaluation of the patients numerous physiological systems, e.g. the pulmonary, metabolic, renal and circulatory systems. Much of the information necessary for this evaluation comes from analysis of the patients' blood. Blood samples can be taken from both arteries and veins. Arterial blood can be sampled either by placing an arterial catheter or cannula in the patient, or by performing an arterial puncture with a needle. Venous blood can be sampled from a cannula or a venous puncture at the periphery (peripheral venous blood); from a catheter placed in the vena cava or right atrium (central venous blood), or from a pulmonary arterial catheter placed in the pulmonary artery (mixed venous blood).

Placement of venous and arterial catheters are invasive procedures and generally restricted to specialized/high dependency departments. In addition, catheterization, cannulation or puncture of the arteries instead of the veins increases the risk of complications such as hemorrhage, bleeding, thrombosis, embolism, neurological damage or pseudo-aneurysm formation. Sampling of arterial blood by arterial puncture is generally considered a more difficult procedure than sampling of venous blood through a venous puncture. Consequently, the routine sampling of arterial blood is generally restricted to specialized/high dependency environments. In other wards where patients are acutely omitted e.g. cardiology, abdominal surgery, thoracic surgery and medicine, routine sampling of peripheral venous blood is most common.

Many of the measurements taken from the blood, and used to assess the patient state, are similar in the venous and arterial blood samples. These includes the electrolytes and metabolites such as sodium (Na), potassium (K), and blood sugar. However, the acid-base status of arterial and venous blood is not the same, regardless of the site of sampling. The acid-base status refers, in general, to the following measurements in blood: the pH, the partial pressure of oxygen (pO2), the partial pressure of carbon dioxide (pCO2), the bicarbonate concentration (HCO3), the hemoglobin concentration (Hb) and the concentration of abnormal forms of hemoglobin (e.g. carboxyhemoglobin (COHb), methylhemoglobin (MetHb), the saturation of hemoglobin with oxygen (SO2), the concentration of base higher than a reference condition (base excess (BE)) and the concentration of bicarbonate at a reference pCO2 (standard bicarbonate SBC). The variation in acid-base status between arterial and venous blood is due to oxygen removal from the blood and carbon dioxide addition due to metabolism at the tissues. In addition, in patients with circulatory or metabolic abnormalities, the production of strong acids at the tissues due to anaerobic metabolism may also modify the acid-base status.

The acid-base status of arterial blood is used to assess the patient's pulmonary and metabolic state. It has been argued (Adrogue et al., 1989a, 1989b; Brandi et al., 1995; Radiometer 1997) and to a large extent clinically accepted that venous blood samples are not adequate for assessing the acid/base and respiratory state of patients. This is thought to be particularly true for peripheral venous samples which “are not recommended for blood gas analysis as they provide little or no information on the general status of the patient” (Radiometer 1997).

In the intensive care unit placement of arterial catheters is routine practice and an assessment of the acid-base status can be obtained from the arterial blood. In some other hospital departments e.g. pulmonary medicine, or nephrology, arterial blood gases are also measured. However, in other wards admitting acutely ill patients, e.g. cardiology, abdominal surgery, thoracic surgery and medicine, arterial samples are not usually taken. Usually a peripheral venous sample is taken and analyzed in a central laboratory. The sample is usually taken aerobically, i.e. no attempt is made to ensure that pO2 and pCO2 remain constant during the sample procedure. Only a small amount of information concerning the acid-base status of the patient is measured in this sample i.e. the standard bicarbonate, SBCv, and hemoglobin Hbv. Other acid base parameters pHv, carbon dioxide pressure (pCO2v), base excess (BEv), oxygen saturation (SO2v) and oxygen pressure (pO2v) are not measured, and if measured would probably not reflect the true values of venous blood at this sample site given the aerobic nature of the sample.

In recent years, methods of converting venous blood gas values to arterial blood gas values have been demonstrated. Over the years, several initiatives have been taken to reduce the need for arterial punctures, for example the method disclosed in international patent application WO 2004/010861 (to OBI Medical Aps, Denmark) for converting venous blood values to arterial blood values. This has the advantage that arterial blood samples need not be taken, and the disadvantages compared to venous blood samples when taking arterial blood samples are then eliminated. The method is essentially based on three steps, namely the first step of measuring arterial oxygenation saturation, e.g. by pulse oximetry, the second step of measuring, preferably by anaerobic sampling, and estimating values of venous blood acid/base status and oxygenation status of a venous blood sample, including peripheral venous blood (PVBG) or central venous blood (CVBG), and the third step of converting the venous blood values by applying a mathematical model for deriving blood acid/base status and oxygenation status into the desired estimated arterial blood values, i.e. one or more values of the acid-base status in the arterial blood. The method described generally in WO 2004/010861 is now commercially available from OBI, A Roche company, under the trade name v-TAC™, cf. the web-page https://diagnostics.roche.com/global/en/products/instruments/v-tac-standalone-ins-6779.html for further information.

As described above, current methods require the provision of a venous blood sample and a measured or estimated arterial oxygenation saturation value (SpO2) from the subject, such as by a pulse oximeter. The v-TAC algorithm then processes the venous blood gas values, the arterial oxygenation saturation value and provides arterial blood gas values. In some instances, an arterial oxygenation value is not present, may be subject to a measurement error, or incorrect reading from a health care person reading and inputting said arterial oxygenation value from said pulse oximeter.

Thus, an improved method converting venous blood values to arterial blood values would be advantageous, and in particular a more efficient and/or reliable method would be advantageous.

### OBJECT OF THE INVENTION

It is a further object of the present invention to provide an alternative to the prior art.

In particular, it may be seen as an object of the present invention to provide a method of providing arterial blood gas values converted from a venous blood value without the provision of a measured or estimated arterial oxygenation saturation value (SpO2), that solves the above mentioned problems of the prior art.

### SUMMARY OF THE INVENTION

Thus, the above described object and several other objects are intended to be obtained in a first aspect of the invention by providing a computer-implemented method of converting venous blood gas values to arterial blood gas values of a subject, if a measured or estimated arterial oxygenation saturation value (SpO2) for the subject is not available, the method comprising:
a. providing a predefined default arterial oxygenation saturation value as a substitute value I,
b. optionally adjusting the substitute value I by an input from a user,
c. providing venous blood gas values of a provided venous blood sample from the subject,
d. applying a mathematical model to the venous blood gas values and the substitute value I, and
e. providing estimated arterial blood gas values based on the mathematical modelling applied to the values provided in step c and the substitute value I.

The invention is particularly, but not exclusively, advantageous for providing a method for converting venous blood gas values to arterial blood gas values without the provision of a measured or estimated arterial oxygenation saturation value (SpO2). Thus, the present invention, eliminates the prerequisite of providing an estimated or measured arterial oxygenation saturation value to perform a conversion, thus simplifying the task of converting arterial blood gas values from venous blood gas values and further to ensure, that at least arterial values including but not limited to pH, pCO2, BE, HCO3, tO2 and tCO2 can be estimated even though a measured or estimated arterial oxygenation saturation value can not be provided, assuming that venous blood gas values such as pH, pCO2, pO2, sO2 and Hb are available.

It is to be understood that venous blood gas values may be derived from a peripheral venous blood sample.

It is further to be understood, that the present invention is enabled to provide estimated arterial blood gas values even without the provision of neither a measured nor an estimated arterial oxygenation value is available.

Even further, it is to be understood, that the mathematical model may, at least partly be a version of the v-TAC algorithm as described in the cited prior art.

Another advantage of the present invention is the reduced chance of measurement error, as the step of providing an arterial oxygenation saturation value has been eliminated from the method of converting arterial blood gas values from venous blood gas values.

Yet another advantage of the present invention is the reduced chance of a reading or input error, when a health care person translates or transfers an estimated or measured arterial oxygenation saturation value, such as from a pulse oximeter to the input of a device or computer program product suitable for converting venous blood gas values and arterial oxygenation saturation values to arterial blood gas values, as the step of providing an arterial oxygenation saturation value has been eliminated from the method of converting arterial blood gas values from venous blood gas values.

Thus, the present invention provides a computer-implemented method for providing arterial blood gas values from a subject, such as a patient, to a user, such as a physician or other health care person, without the provision of an arterial oxygenation saturation value and an arterial blood sample or arterial blood gas values. By reducing the need for the abovementioned arterial oxygenation saturation value and an arterial blood sample, distress and pain to patients, complexity of patient care to any personnel involved, and the risk of error is greatly reduced.

In the context of the present invention, the method is provided for a specific subject, wherein an arterial oxygenation saturation value is not available for said specific subject.

Further, in the context of the present invention, predefined default is to be understood as a value which is not based on any previous estimated, measured or in other ways assessed information with respect to the specific subject.

In the context of the present invention, it is to be understood that providing blood values from a blood sample does not necessarily include the specific step of taking or extracting a blood sample from a patient, thus measurements results may be obtained, transferred, communicated etc. from another entity or person, e.g. a nurse, having performed a blood measurement or extraction.

In an embodiment of the invention, provided estimated arterial blood gas values in step e excludes arterial pO2, as a measured or estimated arterial oxygenation saturation value is not provided.

In a preferred embodiment of the invention, the venous blood gas values of step c are at least one of venous acid/base parameters and venous oxygenation parameters.

In another preferred embodiment, the estimated arterial blood gas values of step e are at least one of arterial oxygenation parameters and arterial acid-base status parameters.

In an embodiment of the invention, the substitute value I of step a is based on clinical/medical guidelines, such as global health guidelines, national health guidelines, regional guidelines, hospital guidelines or physicians guidelines.

In an advantageous embodiment of the invention, the user input of optional step b is based on one or more of whether the subject is currently treated with supplemental oxygen, and/or physical parameters of the subject. The physical parameters may comprise one or more the following: age, pathology/disease, gender, weight, and a user estimated fat percentage.

In the context of the present invention, relevant pathologies/diseases may be one or more of, but not limited to the following: chronic obstructive pulmonary disease (COPD), such as interstitial lung disease (ILD), such as Cystic fibrosis (CF), such as pulmonary hypertension, such as patients with neuromuscular or chest wall disorders or such as patients with advanced cardiac failure.

Further, in the context of the present invention, a user estimated fat percentage is to be understood as a visual assessment or other estimation performed by a health care person during the provision of the input according to optional step b of the present invention.

In an embodiment of the invention wherein the subject receives supplemental oxygen treatment, the user input of step b may be based on whether the subject has received long-term oxygen treatment or acute oxygen treatment. In the context of the present invention, long-term oxygen treatment is to be understood as oxygen treatment for at least 12 hours per day for more than 30 days. It is further to be understood, that acute oxygen treatment may be an acute treatment of a patient in relation to trauma or a sudden onset of a pathology requiring emergent care. It should be noted that a person skilled in the art would know the difference between acute and long-term oxygen treatment.

In another advantageous embodiment of the invention, step c further comprise the provision of haemoglobin values of the provided venous blood sample from the subject, and step d further comprise:
applying the mathematical model to the provided haemoglobin values, and wherein the estimated arterial acid-base status values and blood gas values provided in step e is further based on the mathematical modelling of the haemoglobin values.

In a preferred embodiment of the invention, the mathematical model in step d further applies that a true value of respiratory quotient (RQ) can only vary between 0.7-1.0, being 0.7 in aerobic metabolism of fat and 1.0 in aerobic metabolism of carbohydrate.

In another preferred embodiment of the invention, the mathematical model in step d further mathematically applies:
adding O2 and removing CO2 from the venous blood at a ratio determined by a constant respiratory quotient (RQ) set to be within the physiologically possible range 0.7-1.0, such as at a fraction of RQ set at 0.82, and
performing a simulation until the estimated arterial blood gas values correlates to the provided venous blood gas values and the substitute value I of step a or step b.

In an advantageous embodiment of the invention, the method further comprises providing a machine-learning algorithm, and after step e, the further steps of:
f: providing a measured arterial oxygenation value of the subject,
g: comparing the substitute value I of step a and/or step b to the measured arterial oxygenation value, and
h: adapting subsequent substitute values I of step a, based on at least the comparison performed in step g by the learning algorithm.

This embodiment is particularly advantageous for the method of converting venous blood gas values to arterial blood gas values to continuously improve, based on empirical data.

In an embodiment of the invention, the substitute value I of step a is an arterial oxygen saturation fraction between 0.85 and 1.00.

In the context of the present invention, fraction is to be understood as a decimal fraction wherein 1.00 represents 100% arterial oxygen saturation and wherein 0.85 represents 85% arterial oxygen saturation of a subject. It should be noted that a person skilled in the art would know how these values translate.

In a second aspect, the invention relates to a system adapted to convert venous blood gas values to arterial blood gas values of a subject, if a measured or estimated arterial oxygenation saturation value for the subject is not available, the system comprising:
a user interface, such as a touch screen, the user interface being configured to provide information to a user and to receive inputs from said user,
an input/output device configured to receive data from a peripheral device, such as a blood gas analysing system or device,
a processor configured to process data and employ algorithms, mathematical blood gas models or simulations, preferably wherein the processor is configured to employ the mathematical model according to claim 1,
wherein the system is configured to provide estimated arterial blood gas values to the user, when the system is provided with:
a venous blood gas value from a venous blood sample from the subject, and
a substitute value I, wherein I is predefined default oxygenation saturation value, or
the user inputs a substitute value I representing an oxygenation saturation value.

In the context of the present invention, a user interface is to be understood as any device configured to display a user interface and configured to receive input from a user to be received in a digital device, such as a computer with a screen, keyboard and mouse or a smartphone, tablet or other suitable device. In preferred embodiments, the digital device or system further comprises a memory configured to store data and/or one or more computer program products.

In the context of the present invention, a processor is to be understood as any suitable type of logic circuitry that responds to and processes the basic instructions and data provided to said processor, such as a CPU in a computer configured to execute a computer program product, in particular such as the computer implemented method according to the first aspect of the invention.

In the context of the present invention, input/output device is to be understood as any suitable device adapted to receive/send inputted, outputted or other processed data between the system and a peripheral device, such a blood gas analyser. It may further be adapted acquire respective media data as input sent to a computer or send computer data to storage media as storage output. The input output device may be wired or wireless, such as configured to receive/send data through a wired connection, such a data cable or wirelessly, such as through radio signals.

In a preferred embodiment of the invention, the system is a decision support system, the system being configured to provide the user with decision support regarding the subject, such as decision support with respect to the flow of oxygen from a supplemental oxygen device to the patient. This embodiment is particularly advantageous for obtaining decision support when adjusting oxygen flow to a patient from a supplementary oxygen device. The decision support can assist health care personnel, such as a nurse or physician to, with fewer than usual adjustments of the oxygen flow, reach a desired oxygen level of the patient. The fewer than usual adjustments saves time for the health care personnel and furthermore reduces the amount of time at which the patient is in discomfort.

In a third aspect, the invention relates to a computer program product being adapted to enable a computer system, preferably a portable computer system, comprising at least one computer having data storage means in connection therewith and comprising instructions which, when the program is executed by a computer, to cause the computer to carry out the computer-implemented method of the first aspect of the invention.

This aspect of the invention is particularly, but not exclusively, advantageous in that the present invention may be accomplished by the computer program product enabling a computer system to carry out the operations of the computer implemented method of the first aspect of the invention when down- or uploaded into the computer system. Such a computer program product may be provided on any kind of computer readable medium, or through a network.

In a fourth aspect, the invention relates to the use of the system according to the second aspect of the invention, such as wherein a user adjusts a supplemental oxygen flow to a patient based on the estimated blood gas values provided by the system.

In another embodiment of the invention, the use of the system relates to the use of the decision support system according the second aspect of the invention, such as wherein a user adjusts a supplemental oxygen flow to a patient based decision support provided by the system based on estimated arterial blood gas values of a patient.

The individual aspects of the present invention may each be combined with any of the other aspects. These and other aspects of the invention will be apparent from the following description with reference to the described embodiments.

### BRIEF DESCRIPTION OF THE FIGURES

The computer-implemented method, system and computer program product according to the invention will now be described in more detail with regard to the accompanying figures. The figures show one way of implementing the present invention and is not to be construed as being limiting to other possible embodiments falling within the scope of the attached claim set.

FIG. 1 is a schematic illustration of the method, according to an embodiment of the invention.

FIG. 2 is another schematic illustration of the method, according to an embodiment of the invention.

FIG. 3 is a schematic flow-chart representing an out-line of the operations of the computer program product according to an embodiment of the invention.

### DETAILED DESCRIPTION OF AN EMBODIMENT

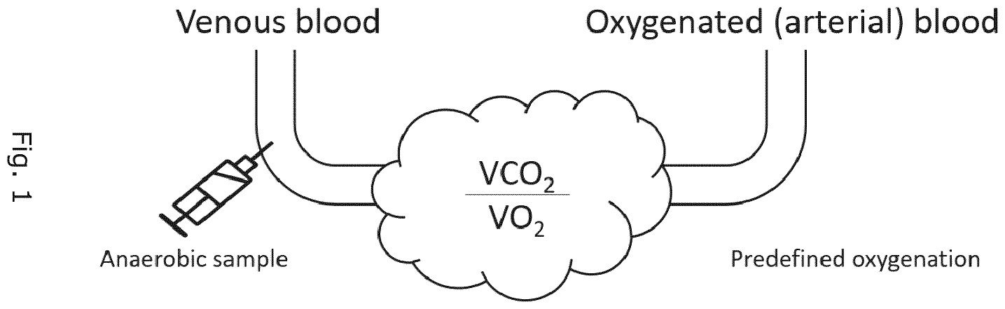

FIG. 1 shows a schematic illustration of the method, according to an embodiment of the invention.

An anaerobic blood sample is obtained from a specific subject, for which specific subject an arterial oxygenation saturation value is not available. The blood sample is analysed with the use of an associated blood gas analyser (not shown). The blood gas analyser provides anaerobic blood gas values, which are provided to the mathematical model. The mathematical model converts the anaerobic blood gas values and provides the predefined default oxygenation saturation value, as substitute value I, and provides estimated/calculated aerobic blood gas values to a user, such as a physician or other health care person based on said anaerobic blood gas values and the substitute value I. It is to be understood, that an anaerobic blood sample may be a venous blood sample and aerobic blood gas values may be arterial blood gas values.

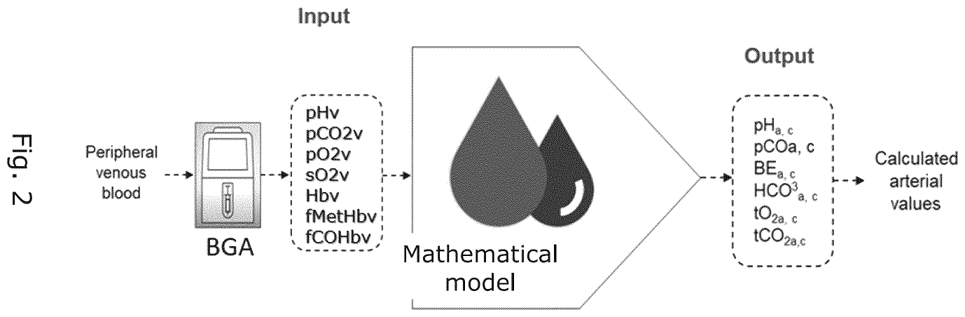

FIG. 2 shows another schematic illustration of the method, according to an embodiment of the invention. A peripheral venous blood sample is provided from a specific subject, for which an arterial oxygenation saturation value is not available. The peripheral venous blood sample is analysed, using a blood gas analyser BGA, and venous blood gas values from the blood gas analyser BGA, such as pH, pCO2, pO2, sO2, Hb, fMETHb and fCOHbv, is input into the mathematical model, preferably a VTAC algorithm. The mathematical model then converts the venous blood gas values and a predefined default arterial oxygenation saturation value, substitute value I, into an output representing calculated arterial blood gas values, such as pH, PCO2, BE, HCO3, tO2 and tCO2.

It is to be understood that for inputs, the v represents venous and the a, c of the outputs represents arterial and calculated respectively.

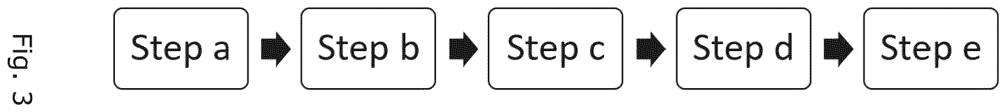

FIG. 3 is a schematic flow-chart representing an out-line of the operations of the computer program product according to an embodiment of the invention. The flow-chart shows the computer implemented method providing a computer-implemented method of converting venous blood gas values to arterial blood gas values of a subject, if a measured or estimated arterial oxygenation saturation value (SpO2) for the subject is not available, the steps of the method comprising:
a. providing a predefined default arterial oxygenation saturation value as a substitute value I,
b. optionally adjusting the substitute value I by an input from a user,
c. providing venous blood gas values of a provided venous blood sample from the subject,
d. applying a mathematical model to the venous blood gas values and the substitute value I, and
e. providing estimated arterial blood gas values based on the mathematical modelling applied to the values provided in step c and the substitute value I.

The invention can be implemented by means of hardware, software, firmware or any combination of these. The invention or some of the features thereof can also be implemented as software running on one or more data processors and/or digital signal processors.

Arterial blood gases are, as an example for a specific subject, estimated as given in the 4 steps below.

Step 1: An anaerobic venous blood sample is drawn from a subject and analysed using standard blood gas analysis technology to provide values of the acid/base and/or blood gas of the venous blood (pH, pCO2, pO2, sO2, Hbv, METHb, COHb).

Step 2: As an arterial oxygenation saturation value is not available for the specific subject, a predefined default arterial oxygenation saturation value as a substitute value I is provided.

Step 3: For a blood sample passing through the tissues from the arteries into the veins, the ratio of the amount of CO₂ added (i.e. the rate of CO₂ production (VCO₂)) and O₂ removed (i.e. the rate of O₂ utilisation (VO₂)), due to aerobic metabolism is defined as the respiratory quotient (RQ=VCO₂/VO₂). RQ is often approximated by measurement of inspiratory and expiratory gases taken at the mouth, through the measurement of inspiratory oxygen (FiO₂) and carbon dioxide (FiCO₂) fraction and either end tidal fractions of oxygen (Fe′O₂) and carbon dioxide (Fe′CO₂) or mixed expired fractions of oxygen (FeO₂) and carbon dioxide (FeCO₂) using the equations:

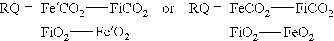

Approximation of RQ by this method often gives values which can vary substantially. However, the true value of RQ at the tissues can only vary between 0.7-1.0, being 0.7 in aerobic metabolism of fat and 1.0 in aerobic metabolism of carbohydrate. In this step a mathematical model of blood acid/base and oxygenation status (e.g. Rees et al, 1996, 1997, etc.) is used to perform a simulation, where O₂ is added and CO₂ removed from the venous blood in a ratio determined by a constant respiratory quotient, set to be within the physiologically possible range 0.7-1.0. This simulation is performed until the simulated oxygen saturation is equal to the substitute value I of step 2.

Step 4: The model of blood acid/base and substitute value I is then used to calculate an estimate of arterial acid/base status of the arterial blood. This is possible as the simulated removal of CO2 and O2 from venous blood at a fixed RQ ensures that when the simulated arterial oxygenation matches substitute value I, then the simulated values of other arterial acid-base variables should also match the venous values provided.

The fundamental assumption contained in this method is that a constant value of RQ may be used to perform the venous arterial conversion. This requires that little or no anaerobic metabolism occurs across the tissue where the venous blood sample is taken. If anaerobic metabolism were present the strong acid produced by this process (H⁺) would bind with bicarbonate (HCO3⁻) in the blood to form CO₂ in the following reversible reaction

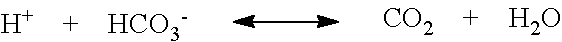

The increase in CO₂ production by this reaction would mean that the apparent VCO₂ would be increased without an increase in VO₂, and hence RQ would increase. The degree of anaerobic metabolism depends upon the circulatory and metabolic state of the patient. In a normal well perfused peripheral limb it is unlikely that anaerobic metabolism occurs. The quality of perfusion of a limb can be assessed clinically by the presence of a clearly recognizable arterial pulse determined by palpation, a normal capillary response, and a normal color and temperature of the limb. Central or mixed venous blood is a mixture of blood from several sites and may therefore contain blood from an area of the body with anaerobic metabolism. The selection of the sample site is therefore important.

Step 5: Output of arterial values such as pH, pCO2, BE, HCO3, tO2 and tCO2. It should be noted, that as a measured or estimated arterial oxygenation saturation value is not provided, the method is currently not able to provide arterial pO2 as within the cited prior art, but is enabled to provide the abovementioned arterial values which may be valuable to a physician or other health care person.

A simulation, wherein the present invention has been used, has been performed on data from the following three clinical studies:

Ekström M et al. (2019). Calculated arterial blood gas values from a venous sample and pulse oximetry: Clinical validation. PLOS ONE 14 (4): e0215413. doi: 10.1371/journal.pone.0215413

Rees S E et al. (2012). Calculating acid-base and oxygenation status during COPD exacerbation using mathematically arterialised venous blood. Clin Chem Lab Med 50 (12): 2149-2154. doi: 10.1515/cclm-2012-0233

Tygesen G et al. (2012). Mathematical arterialization of venous blood in emergency medicine patients. Eur J Emerg Med 19:363-372. doi: 10.1097/MEJ.0b013e32834de4c6

A total of n=472 data sets were used in the simulation.

Each data set consists of an arterial blood gas measurement (ABG) used as reference and a venous blood gas measurement (VBG) with associated SpO2 measurement from pulse oximeter.

A simulation was conducted where the VBGs were converted to arterial values using:
Measured SpO2 for each data set.
90% constant for all data sets, as a substitute value I.
94% constant for all data sets, as a substitute value I.

The VBG and the three converted results were compared to the ABG reference using statistical methods to calculate:
Mean bias (average difference).
95% Limits of Agreement, calculated as 1.96*SD.

The results are listed in tables presented below, for pH and pCO2 respectively.

pH results vs ABG (reference):

| | Mean bias | 95% LoA |
|---|---|---|
| VBG | 0.0302 | 0.049 |
| v-TAC @SpO2 | 0.0002 | 0.028 |
| present invention @94% | 0.0005 | 0.031 |
| present invention @90% | 0.0046 | 0.030 |

pCO2 results vs ABG (reference):

| | Mean bias | 95% LoA |
|---|---|---|
| | [kPa] | [kPa] |
| VBG | −0.8132 | 1.211 |
| v-TAC @SpO2 | −0.0564 | 0.507 |
| present invention @94% | −0.0552 | 0.563 |
| present invention @90% | −0.1549 | 0.560 |

The simulations show, that the present invention is able to convert venous pH and pCO2 (but not pO2) values to arterial values using a predefined, patient-independent constant with an accuracy and precision that is close to using a measured SpO2 for each individual data set and significantly better than using VBG values alone without conversion by the present invention.

In short, the present invention relates to a computer-implemented method, system and decision support system adapted to provide arterial venous blood gas values without the provision of an arterial oxygenation saturation value or arterial blood gas values. The method comprises the provision of arterial blood gas values from a subject, for which said subject, only venous blood gas values are provided, by providing a mathematical model adapted to convert said venous blood gas values with a provided predefined default arterial oxygenation value to output arterial blood gas values of said subject. The present invention thus provides a method for providing arterial blood gas values from a specific subject without the need of providing an arterial blood sample from a painful arterial blood draw or the need for an arterial oxygenation saturation value of the subject, thus reducing distress to said patient and a reduction of tasks to relevant health care personnel.

In the following preferred embodiments and aspects of the invention are presented as a list of items:

Item 1. A computer-implemented method of converting venous blood gas values to arterial blood gas values of a subject, if a measured or estimated arterial oxygenation saturation value for the subject is not available, the method comprising:
a. providing a predefined default arterial oxygenation saturation value as a substitute value I,
b. optionally adjusting the substitute value I by an input from a user,
c. providing venous blood gas values of a provided venous blood sample from the subject,
d. applying a mathematical model to the venous blood gas values and the substitute value I, and
e. providing estimated arterial blood gas values based on the mathematical modelling applied to the values provided in step c and the substitute value I.

Item 2. The computer-implemented method according to Item 1, wherein the venous blood gas values of step c are at least one of venous acid/base parameters and venous oxygenation parameters.

Item 3. The computer-implemented method according to Item 1 or 2, wherein the arterial blood gas values of step e are at least one of arterial oxygenation parameters and arterial acid-base status parameters.

Item 4. The computer-implemented method according to any of the preceding Items, wherein the substitute value I of step a is based on clinical/medical guidelines, such as global health guidelines, national health guidelines, regional guidelines, hospital guidelines or physicians guidelines.

Item 5. The computer-implemented method according to any of the preceding Items, wherein the user input of optional step b is based on one or more of:
if the subject is currently treated with supplemental oxygen, and/or
physical parameters of the subject, the physical parameters comprising one or more of:
   age,
   pathology/disease, such as COPD,
   gender,
   weight, and
   a user estimated fat percentage.

Item 6. The computer-implemented method according to any of the preceding Items, wherein step c further comprises:
providing haemoglobin values of the provided venous blood sample from the subject, and
step d further comprises:
applying the mathematical model to the provided haemoglobin values, and wherein the estimated arterial acid-base status values and blood gas values provided in step e is further based on the mathematical modelling of the haemoglobin values.

Item 7. The computer-implemented method according to any of the preceding Items, wherein the mathematical model in step d further applies that a true value of respiratory quotient (RQ) can only vary between 0.7-1.0, being 0.7 in aerobic metabolism of fat and 1.0 in aerobic metabolism of carbohydrate.

Item 8. The computer-implemented method according to any of the preceding Items, wherein the mathematical model in step d further mathematically applies:
adding O2 and removing CO2 from the venous blood at a ratio determined by a constant respiratory quotient (RQ) set to be within the physiologically possible range 0.7-1.0, such as at a fraction of RQ set at 0.82, and
performing a simulation until the estimated arterial blood gas values correlates to the provided venous blood gas values and the substitute value I of step a or step b.

Item 9. The computer-implemented method according to any of the preceding Items, the method further comprising providing a machine learning algorithm, and after step e, the further steps of:
f: providing a measured arterial oxygenation value of the subject,
g: comparing the substitute value I of step a and/or step b to the measured arterial oxygenation value, and
h: adapting subsequent substitute values I of step a, based on at least the comparison performed in step g by the learning algorithm.

Item 10. The computer-implemented method according to Item 1, wherein the substitute value I of step a is an arterial oxygen saturation fraction between 0.85 and 1.00.

Item 11. A system adapted to convert venous blood gas values to arterial blood gas values of a subject, if a measured or estimated arterial oxygenation saturation value for the subject is not available, the system comprising:
a user interface, such as a touch screen, the user interface being configured to provide information to a user and to receive inputs from said user,
an input/output device configured to receive data from a peripheral device, such as a blood gas analysing system or device,
a processor configured to process data and employ algorithms, mathematical blood gas models or simulations, preferably wherein the processor is configured to employ the mathematical model according to Item 1,
wherein the system is configured to provide estimated arterial blood gas values to the user, when the system is provided with:
a venous blood gas value from a venous blood sample from the subject, and
a substitute value I, wherein I is predefined default oxygenation saturation value, or
the user inputs a substitute value I representing an oxygenation saturation value.

Item 12. The system according to Item 11, wherein the system is a decision support system, the system being configured to provide the user with decision support regarding the subject, such as decision support with respect to the flow of oxygen from a supplemental oxygen device to the patient.

Item 13. The decision support system according to Item 12, the decision support system further adapted to adjust a supplemental oxygen flow to a subject, based on one or more user inputs, wherein the user has received decision support with respect to said supplemental oxygen flow, from the decision support system.

Item 14. A computer program product enabling a computer system, preferably a portable computer system, to carry out the method according to Item 1, when down- or uploaded into the computer system.

Item 15. Use of the system according to Item 11, wherein a user adjusts a supplemental oxygen flow to a patient based on the estimated blood gas values provided by the system.

Item 16. A method of treating oxygen deficiency in a subject receiving supplemental oxygen, the method comprising
executing the steps according to Item 1,
determining if an estimated arterial blood gas value is within a threshold range, and if the estimated arterial blood gas value is outside said threshold range,
treating said subject based on the estimated arterial blood gas values, such as by adjusting the rate of supplemental oxygen per minute.

Item 17. A pulmonary ventilation device adapted to ventilate a subject, the device comprising:
a ventilator,
a processor adapted to execute a mathematical model, the mathematical model enabling conversion of venous blood gas values and SpO2 values into estimated ABG values,
an input interface in data connection with the processor, the input interface adapted to receive at least said venous blood gas and SpO2 values measured from the subject,
a user interface,
a controller in data connection with the processor and ventilator, the controller adapted to control and adjust the ventilator based on the estimated arterial blood gas values from the processor,
wherein, if SpO2 values are not received from the subject, the device executes the computer implemented method according to Item 1.

Item 18. The device according to Item 17, wherein the device further alerts a user as to the non-received SpO2 values, the implementation of the substitute value I and numerical value of I, to provide the estimated ABG values required for the device to ventilate the subject, and optionally inquire approval from the user regarding the numerical value of I.

Item 19. The computer-implemented method according to Item 1, wherein the provided estimated arterial blood gas values of step e consists of pH, pCO2, BE, HCO3, tO2 and tCO2.

Item 20. The computer-implemented method according to Item 1, wherein the provided estimated arterial blood gas values of step e consists of one or more of: pH, pCO2, BE, HCO3, tO2 and tCO2.

In the context of the present invention, the following definitions and abbreviations may be used:

| Abbreviation | Meaning |
|---|---|
| Hb | The haemoglobin level of the venous or the capillary blood, |
| | measured by a blood gas analyser or by other means |
| pH | The venous or arterial pH value |
| pO2 | The partial pressure of oxygen of venous or capillary blood, |
| | measured by a blood gas analyser or by other means |
| pCO2 | The partial pressure of carbon dioxide of venous or arterial |
| | blood |
| sO2 | The oxygen saturation level of the venous or the capillary |
| | blood, measured by a blood gas analyser or by other means |
| HCO3 | The estimated bicarbonate level of arterial blood |
| BE | The base excess refer to an excess, in the amount of base |
| | present in the blood. The value is usually reported as a |
| | concentration in units of mEq/L (mmol/L), with positive |
| | numbers indicating an excess of base. |
| | Even further, base excess is defined as the amount |
| | of strong acid that must be added to each liter |
| | of fully oxygenated blood to return the |
| | pH to 7.40 at a temperature of 37° C. |
| | and a pCO2 of 40 mmHg (5.3 kPa). |
| v-TAC ™ | The v-TAC ™ software, based on the patented method for |
| | mathematical arterialisation of venous blood, cf. WO |
| | 2004/010861 (to OBI Medical Aps, Denmark), |
| | which is hereby incorporated by reference in its entirety. |
| VTAC | Any mathematical algorithm, equation, formula, model, |
| | computer program or mechanism, such as but not limited |
| | to the v-TAC ™ software, for calculation of |
| | ABG_{P} values from blood, such as but not |
| | limited to venous or capillary blood |
| tO2 | The total oxygen content of venous or arterial blood |
| tCO2 | The total carbon dioxide content of venous or arterial blood |
| fMetHb | The fraction of measured venous hemoglobin in |
| | the form of metalloprotein, i.e. methaemoglobin, |
| | in which the iron in the heme group |
| | is in the Fe3+ (ferric) state, |
| | not the Fe2+ (ferrous) of normal |
| | haemoglobin. Sometimes, it is also |
| | referred to as ferrihemoglobin |
| fCOHb | The fraction of carboxyhemoglobin of the venous, capillary |
| | blood or arterial blood, measured by a blood |
| | gas analyser or by other means |
| SpO2 | Arterial saturation level measured by pulse oximetry |
| 95% LoA | 95% limits of agreement, Bland-Altman plot |

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

The invention claimed is:

1. A computer-implemented method of converting venous blood gas values to arterial blood gas values of a subject, if a measured arterial oxygenation saturation value for the subject is not provided, the method comprising:
a. providing a predefined default arterial oxygenation saturation value as a substitute value I that is based on clinical/medical guidelines comprising at least one of global health guidelines, national health guidelines, regional guidelines, hospital guidelines or physicians guidelines;
b. adjusting the substitute value I by an input from a user;
c. providing venous blood gas values of a provided venous blood sample from the subject;
d. applying a mathematical model to the venous blood gas values and the substitute value I; and
e. providing estimated arterial blood gas values based on the mathematical modelling applied in step d, the provided estimated arterial blood gas values selected from one or more of pH, pCO2, BE, HCO3, tO2 and tCO2.

2. The computer-implemented method according to claim 1, wherein the venous blood gas values of step c are at least one of venous acid/base parameters and venous oxygenation parameters.

3. The computer-implemented method according to claim 1, wherein the arterial blood gas values of step e are at least one of arterial oxygenation parameters and arterial acid-base status parameters.

4. The computer-implemented method according to claim 1, wherein the user input of optional step b is applied, and wherein the user input is based on at least one of:
if the subject is currently treated with supplemental oxygen or;
physical parameters of the subject, the physical parameters comprising at least one of age, pathology, disease, gender, weight and a user estimated fat percentage.

5. The computer-implemented method according to claim 1, wherein step c further comprises providing haemoglobin values of the provided venous blood sample from the subject, and step d further comprises applying the mathematical model to the provided haemoglobin values, and wherein the estimated arterial acid-base status values and blood gas values provided in step e is further based on the mathematical modelling of the haemoglobin values.

6. The computer-implemented method according to claim 1, wherein the mathematical model in step d further applies that a true value of respiratory quotient (RQ) can only vary between 0.7-1.0, being 0.7 in aerobic metabolism of fat and 1.0 in aerobic metabolism of carbohydrate.

7. The computer-implemented method according to claim 1, wherein the mathematical model in step d further mathematically applies:
adding O2 and removing CO2 from the venous blood at a ratio determined by a constant respiratory quotient (RQ) set to be within the physiologically possible range 0.7-1.0; and
performing a simulation until the estimated arterial blood gas values correlates to the provided venous blood gas values and the substitute value I of step a or step b.

8. The computer-implemented method according to claim 1, the method further comprising providing a machine learning algorithm, and after step e, the further steps of:
f: providing a measured arterial oxygenation value of the subject;
g: comparing the substitute value I of at least one of step a or step b to the measured arterial oxygenation value; and
h: adapting subsequent substitute values I of step a, based on at least the comparison performed in step g by the learning algorithm.

9. The computer-implemented method according to claim 1, wherein the substitute value I of step a is an arterial oxygen saturation fraction between 0.85 and 1.00.

10. A system adapted to convert venous blood gas values to arterial blood gas values of a subject when a measured arterial oxygenation saturation value for the subject is not provided, the system comprising:
a user interface configured to provide information to a user and to receive inputs from the said user;
an input/output device configured to receive data from a peripheral device, comprising a blood gas analysing system or device;
a processor configured to process data and employ algorithms, mathematical blood gas models or simulations, preferably wherein the processor is configured to employ the mathematical model according to claim 1;
wherein the system is configured to provide estimated arterial blood gas values to the user, when the system is provided with:
a venous blood gas value from a venous blood sample from the subject; and
a substitute value I representing a predefined default oxygenation saturation value that is based on clinical/medical guidelines comprising at least one of global health guidelines, national health guidelines, regional guidelines, hospital guidelines or physicians guidelines; or
the user inputs a substitute value I representing an oxygenation saturation value, the provided estimated arterial blood gas values selected from one or more of pH, pCO2, BE, HCO3, tO2 and tCO2.

11. The system according to claim 10, wherein the system is a decision support system, the system being configured to provide the user with decision support with respect to the flow of oxygen from a supplemental oxygen device to the subject.

12. The decision support system according to claim 11, the decision support system further adapted to adjust a supplemental oxygen flow to a subject, based on one or more user inputs, wherein the user has received decision support with respect to the supplemental oxygen flow, from the decision support system.

13. A computer program product enabling a computer system to carry out the method according to claim 1, when down- or uploaded into the computer system.

14. Use of the system according to claim 10, wherein a user adjusts a supplemental oxygen flow to a patient based on the estimated arterial blood gas values provided by the system.

15. A method of treating oxygen deficiency in a subject receiving supplemental oxygen, the method comprising
executing the steps according to claim 1;
determining if an estimated arterial blood gas value is within a threshold range, and if the estimated arterial blood gas value is outside the threshold range; and
treating the subject based on the estimated arterial blood gas values by adjusting the rate of supplemental oxygen per minute.

16. A pulmonary ventilation device adapted to ventilate a subject, the device comprising:
a ventilator;
a processor adapted to execute a mathematical model, the mathematical model enabling conversion of venous blood gas values and SpO2 values into estimated ABG values;
an input interface in data connection with the processor, the input interface adapted to receive at least the venous blood gas and SpO2 values measured from the subject;
a user interface; and
a controller in data connection with the processor and ventilator, the controller adapted to control and adjust the ventilator based on the estimated arterial blood gas values from the processor, wherein, if SpO2 values are not received from the subject, the device executes the computer implemented method according to claim 1.

17. The device according to claim 16, wherein the device further alerts a user as to the non-received SpO2 values, the implementation of the substitute value I and numerical value of I, to provide the estimated ABG values required for the device to ventilate the subject, and optionally inquire approval from the user regarding the numerical value of I.

18. The computer-implemented method according to claim 1, wherein the provided estimated arterial blood gas values of step e does not include an estimated arterial pO2 value.

19. The computer-implemented method according to claim 4, wherein the disease comprises COPD.

20. The computer-implemented method according to claim 7, wherein the adding O2 and removing CO2 from the venous blood occurs at a fraction of RQ set at 0.82.
